(19) United States
(12) Patent Application Publication
(10) Pub. No.: US 2019/0183138 A1
(43) Pub. Date: Jun. 20, 2019

(54) MILK COAGULANT AND METHOD FOR PRODUCING CHEESE

(71) Applicant: CHINA AGRICULTURAL UNIVERSITY

(72) Inventors: Fazheng REN, Beijing (CN); Chen XIAO, Beijing (CN); Hao ZHANG, Beijing (CN); Jie LUO, Beijing (CN); Huiyuan GUO, Beijing (CN)

(21) Appl. No.: 16/109,871

(22) Filed: Aug. 23, 2018

(30) Foreign Application Priority Data
Dec. 19, 2017 (CN) 201711376336.8

(51) Int. Cl. A23C 19/032; C12N 9/50; A23C 19/04

(52) U.S. Cl. CPC A23C 19/0326; A23C 19/041; C12Y 304/22007; C12N 9/63

(57) ABSTRACT

Provided in the present disclosure are a milk coagulant, a method for obtaining asclepain of Asclepias Linn. and cysteine protease B of Calotropis R. Br., as well as a method of producing cheese. The milk coagulant includes at least one of the asclepain of Asclepias Linn. and the cysteine protease B of Calotropis R. Br.

### CROSS-REFERENCE TO RELATED APPLICATIONS

This application is based upon and claims priority to Chinese Patent Application No. 201711376336.8, filed on Dec. 19, 2017, the entire content of which is incorporated herein by reference.

### FIELD

The present disclosure relates to the food field, particular to a milk coagulant and a method for producing cheese, more particular to a milk coagulant, a method for obtaining asclepain of Asclepias Linn. and cysteine protease B of Calotropis R. Br. and a method of producing cheese.

### BACKGROUND

Cheese is a fermented or fresh milk product prepared by curding a raw material (such as, milk, watery cream, partly skimmed milk, buttermilk or their combination) with a milk coagulant like chymosin followed by separating milk serum. The cheese is rich of proteins, fat, vitamins and minerals such as calcium, phosphorus and the like, which is also called as “milk gold”.

However, there is still a need to explore the types of milk coagulant.

### SUMMARY

Embodiments of the present disclosure aim at to solve at least one of problems existing in the related art to at least some extent. For this purpose, the present disclosure in embodiments provides a milk coagulant, a method for obtaining asclepain of Asclepias Linn. and cysteine protease B of Calotropis R. Br., as well as a method of producing cheese. According to embodiments of the present disclosure, at least one of the asclepain of Asclepias Linn. and the cysteine protease B of Calotropis R. Br. as the milk coagulant functions on milk clotting under a broad range of temperatures and at a wide range of pH values, as well exhibits great milk-clotting effect, with the cheese obtained in excellent texture and sensory quality.

It should be noted that the present disclosure is accomplished by present inventors based on the following findings.

Although calf rennet is a chymosin widely used in the manufacture of cheese, the production and application of the calf rennet are limited to many factors. For example, the calf rennet produced by killing a calf is still in short supply due to the increase in cheese demand For another example, the use of animal rennet in cheese production is limited by the diet taboos of religious (such as Judaism, Islam and the like) and vegetarian consumers.

In view of the above, it is found by the present inventors that Cynanchum otophyllum Schneid. (C. otophyllum, Chinese name “Qingyangshen”) exhibits milk-clotting effect. Further, the present inventors extract proteases from roots, stems or leaves of C. otophyllum respectively, and determine individual milk-clotting activities of the proteases obtained, with results showing the milk-clotting activities of the proteases are highest in a leave extracted solution, middle in a stem extracted solution and lowest in a root extracted solution. Furthermore, the proteases from the C. otophyllum leaves are separated and purified by the present inventors, discovering that two proteases QA and QC exhibit milk-clotting effect. The proteases QA and QC are identified to be asclepain of Asclepias Linn. and cysteine protease B of Calotropis R. Br., respectively. With further investigation, it is found by the present inventors that such two proteases function on milk clotting under a broad range of temperatures and at a wide range of pH values, as well exhibit great milk-clotting effect, with the cheese obtained in excellent texture and sensory quality.

In one aspect, the present disclosure in embodiments provides a milk coagulant. According to embodiments of the present disclosure, the milk coagulant includes at least one of asclepain of Asclepias Linn. and cysteine protease B of Calotropis R. Br. It is surprisingly discovered by the present inventors that the asclepain of Asclepias Linn. and the cysteine protease B of Calotropis R. Br. exhibit milk-clotting effect. With further investigation, it is found by the present inventors that such two proteases function on milk clotting under a broad range of temperatures and at a wide range of pH values, as well exhibit great milk-clotting effect, with the cheese obtained in excellent texture and sensory quality.

In embodiments of the present disclosure, the milk coagulant in this aspect also has additional technical features as follows.

In embodiments of the present disclosure, the asclepain of Asclepias Linn. and the cysteine protease B of Calotropis R. Br. are from Cynanchum otophyllum Schneid., preferably leaves of the Cynanchum otophyllum Schneid.

In embodiments of the present disclosure, the milk coagulant further includes at least one of a calcium-containing compound and an aluminum-containing compound, thereby improving milk-clotting activity.

In embodiments of the present disclosure, the milk coagulant functions under a temperature of 40° C. to 70° C. and at a pH value of 5.5 to 8.0. It is discovered by the present inventors that the asclepain of Asclepias Linn. and the cysteine protease B of Calotropis R. Br., respectively achieve optimum milk-clotting effects under such the temperature and at the pH value.

In embodiments of the present disclosure, the asclepain of Asclepias Linn. is capable of hydrolyzing Ser132-Thr133 peptide linkage on κ-casein.

In embodiments of the present disclosure, the cysteine protease B of Calotropis R. Br. is capable of hydrolyzing Asp14-Glu15 peptide linkage and Ser132-Thr133 peptide linkage on κ-casein.

In another aspect, the present disclosure in embodiments provides a method for obtaining asclepain of Asclepias Linn. and cysteine protease B of Calotropis R. Br. According to embodiments of the present disclosure, the method includes: soaking leaves of Cynanchum otophyllum Schneid. in a buffer, followed by collecting an extracted solution; and purifying the extracted solution, so as to obtain the asclepain of Asclepias Linn. and the cysteine protease B of Calotropis R. Br., respectively, wherein the asclepain of Asclepias Linn. and the cysteine protease B of Calotropis R. Br. are those in the milk coagulant as defined in the above aspect. With the method of the present disclosure in this aspect, it is possible to extract the asclepain of Asclepias Linn. and the cysteine protease B of Calotropis R. Br. with high purity by effective and simple operations.

In embodiments of the present disclosure, the method for obtaining the asclepain of Asclepias Linn. and the cysteine protease B of Calotropis R. Br. in the above aspect also has additional technical features as follows.

In embodiments of the present disclosure, the buffer is a citric acid-phosphate buffer, thereby facilitating to dissolve C. otophyllum proteases into the buffer.

In embodiments of the present disclosure, the citric acid-phosphate buffer is of a concentration of 10 mmol/L, thereby further facilitating to dissolve C. otophyllum proteases into the buffer.

In embodiments of the present disclosure, the leaves of Cynanchum otophyllum Schneid. and the buffer are at a ratio of mass to volume from 1:10 to 1:30, for example 1:20, thereby further facilitating to dissolve C. otophyllum proteases into the buffer.

In embodiments of the present disclosure, the leaves of Cynanchum otophyllum Schneid. are soaked under 4° C. to 25° C. for 30 minutes to 50 minutes, preferably under 4° C. for 40 minutes, thereby further facilitating to dissolve C. otophyllum proteases into the buffer.

In embodiments of the present disclosure, purifying the extracted solution further includes: subjecting the extracted solution to ultrafiltration for concentration, thereby obtaining a concentrated solution; and eluting the concentrated solution on a chromatographic column, so as to obtain proteases of Cynanchum otophyllum Schneid., wherein eluting the concentrated solution further includes steps of: 1) loading the concentrated solution onto the chromatographic column and collecting a first outflow, so as to obtain the asclepain of Asclepias Linn.; 2) loading a citric acid-phosphate buffer onto the chromatographic column obtained in step 1), with a second outflow obtained; and 3) loading a citric acid-phosphate buffer containing 0.6 mmol/L NaCl onto the chromatographic column obtained in step 2), and collecting a third outflow, so as to obtain the cysteine protease B of Calotropis R. Br., thereby benefiting for separation, purification and acquisition of the asclepain of Asclepias Linn. and the cysteine protease B of Calotropis R. Br.

In embodiments of the present disclosure, the ultrafiltration for concentration is performed using an ultrafiltration tube in 10.0 kD, thereby further benefiting for separation, purification and acquisition of the asclepain of Asclepias Linn. and the cysteine protease B of Calotropis R. Br.

In still another aspect, the present disclosure in embodiments provides a method of producing cheese. According to embodiments of the present disclosure, the method includes: mixing cheese milk with the milk coagulant as defined in above aspects, thereby obtaining a mixture; and keeping the mixture standing for a time period, so as to obtain the cheese. With the method of producing cheese in embodiments of the present disclosure, the cheese obtained is in excellent texture and sensory quality.

In embodiments of the present disclosure, the method in this aspect further includes: adjusting the mixture obtained to be at a pH value of 5.5 to 8.0, thus the milk coagulant exhibits optimum milk-clotting effect at such the pH value.

In embodiments of the present disclosure, the mixture is kept standing under 40° C. to 70° C. for 40 minutes to 90 minutes, thus the milk coagulant exhibits milk-clotting effect sufficiently under such the temperature.

The additional aspects and advantages of the present disclosure will be given partly from the following description, part of which will become apparent from the description or understood from the practice of the present disclosure.

### BRIEF DESCRIPTION OF THE DRAWINGS

The above and/or additional aspects and advantages of the present disclosure will become apparent and easily understood from the description of embodiments in combination with the accompanying drawings, in which:

FIG. 1 shows a flow chart of a method of obtaining proteases QA and QC according to an embodiment of the present disclosure;

FIG. 2 shows a flow chart of specific steps of purifying the extracted solution according to another embodiment of the present disclosure;

FIG. 3 shows electrophoresis of a concentrated solution (A) and a filtered solution (B) of C. otophyllum proteases according to an embodiment of the present disclosure. For the concentrated solution (A), Lane M: a protein molecular weight marker, Lane 1: an extracted solution of C. otophyllum proteases, Lane 2: the concentrated solution; and for the filtered solution (B), Lane M: a protein molecular weight marker, Lane 1: an extracted solution of C. otophyllum proteases, Lane 2: the filtered solution;

FIG. 4 shows SDS-PAGE electrophoresis of purified components of C. otophyllum proteases according to an embodiment of the present disclosure. Lane M: protein molecular weight marker; Lane U: a concentrated solution of C. otophyllum proteases after ultrafiltration; Lanes QA, QB, QC to QD: protein components QA, QB, QC and QD, respectively;

FIG. 5 shows milk-clotting activity of Cynanchum otophyllum Schneid. proteases, QA, QB, QC, QD, and other proteases according to an embodiment of the present disclosure. Different letters indicate a significant difference between different groups (P<0.05). SU=Soxhlet unit. *The curd was not formed within 4 h. Error bars represent the SD of triplicate experiments.

FIG. 6 shows lineweaver-Burk plots for the hydrolysis of κ-casein by Cynanchum otophyllum Schneid. proteases QA (A) and QC (B) according to an embodiment of the present disclosure.

FIG. 7 shows electrophoresis of hydrolysis whole casein: α-casein (A), β-casein (B), and κ-casein (C) by Cynanchum otophyllum Schneid. proteases QA and QC as a function of time according to an embodiment of the present disclosure. Lane M: a protein molecular weight marker; lane 1: α-casein (A), β-casein (B), and κ-casein (C); lanes 2 to 7: casein hydrolyzed by QA at 5 min, 15 min, 30 min, 1 h, 2 h, and 4 h, respectively; lanes 8 to 13: casein hydrolyzed by QC at 5 min, 15 min, 30 min, 1 h, 2 h, and 4 h, respectively. Frames indicate that the bands are excised from the gel to determine the cleavage site on κ-casein by the proteases.

FIG. 8 shows effect of different pH values on proteolytic activities (PA) of C. otophyllum proteases QA (a) and QC (b) according to an embodiment of the present disclosure;

FIG. 9 shows effect of different temperatures on proteolytic activities of C. otophyllum proteases QA (a) and QC (b) according to an embodiment of the present disclosure;

FIG. 10 shows effect of different protease inhibitors on proteolytic activities of C. otophyllum proteases QA (a) and QC (b) according to an embodiment of the present disclosure;

FIG. 11 shows effect of different pH values on milk-clotting activities (MCA) of C. otophyllum proteases QA (a) and QC (b) according to an embodiment of the present disclosure;

FIG. 12 shows effect of different temperatures on milk-clotting activities of C. otophyllum proteases QA (a) and QC (b) according to an embodiment of the present disclosure;

FIG. 13 shows effect of different metal ions on milk-clotting activities of C. otophyllum proteases QA (a) and QC (b) according to an embodiment of the present disclosure.

### DETAILED DESCRIPTION

The embodiments of the present disclosure are described in detail below. Such embodiments are explanatory, and aim at to explain the present disclosure rather than to be constructed to limit the present disclosure.

The present disclosure provides in embodiments a milk coagulant, a method for obtaining asclepain of Asclepias Linn. and cysteine protease B of Calotropis R. Br., as well as a method of producing cheese, each of which will be described in detail as bellows.

For better understanding, milk-clotting mechanism of the milk coagulant is set forth briefly as follows.

Milk-clotting reaction is performed in two steps in the presence of the milk coagulant. Specifically, in an initial step, κ-casein is hydrolyzed by the milk coagulant to be non-active and degraded into para-k-casein and glycomacropeptide (i.e. a small peptide which is trichloroacetic acid-dissoluble); subsequently, in a second step, the para-k-casein is gradually aggregated to become a three-dimensional network structure in the presence of adequate calcium ions under a temperature higher than 20° C., thus forming a milk-clot.

Milk Coagulant

In one aspect, the present disclosure in embodiments provides a milk coagulant. According to embodiments of the present disclosure, the milk coagulant includes at least one of asclepain of Asclepias Linn. and cysteine protease B of Calotropis R. Br. It is surprisingly discovered by the present inventors that the asclepain of Asclepias Linn. and the cysteine protease B of Calotropis R. Br. exhibit milk-clotting effect. With further investigation, it is found by the present inventors that such two proteases function on milk clotting under a broad range of temperatures and at a wide range of pH values, as well exhibit great milk-clotting effect, with the cheese obtained in excellent texture and sensory quality.

In embodiments of the present disclosure, the asclepain of Asclepias Linn. (i.e. protease QA in short) and the cysteine protease B of Calotropis R. Br. (i.e. protease QC in short) are from Cynanchum otophyllum Schneid., preferably leaves of the Cynanchum otophyllum Schneid. The present inventors extract proteases from roots, stems or leaves of C. otophyllum respectively, and determine individual milk-clotting activities of the proteases obtained, with results showing the milk-clotting activities of the proteases are highest in a leave extracted solution, middle in a stem extracted solution and lowest in a root extracted solution. Further, the proteases from the C. otophyllum leaves are separated and purified by the present inventors, discovering that two proteases QA and QC exhibit milk-clotting effect. The proteases QA and QC are identified to be asclepain of Asclepias Linn. and cysteine protease B of Calotropis R. Br., respectively.

In embodiments of the present disclosure, the milk coagulant further includes at least one of a calcium-containing compound and an aluminum-containing compound. It is discovered by the present inventors that calcium ions and aluminum ions improve the milk-clotting activities of the proteases QA and QC significantly, thus guaranteeing good milk-clotting effect for the QA and QC, for example, reduced milk-clotting time, and the cheese obtained in suitable texture.

In embodiments of the present disclosure, the milk coagulant functions under a temperature of 40° C. to 70° C. and at a pH value of 5.5 to 8.0. It is found by the present inventors that the proteases QA and QC exhibit great milk-clotting effect.

In embodiments of the present disclosure, the asclepain of Asclepias Linn. is capable of hydrolyzing Ser132-Thr133 peptide linkage on κ-casein; and the cysteine protease B of Calotropis R. Br. is capable of hydrolyzing Asp14-Glu15 peptide linkage and Ser132-Thr133 peptide linkage on κ-casein. Both the asclepain of Asclepias Linn. and the cysteine protease B of Calotropis R. Br. are able to hydrolyze α-casein, β-casein and κ-casein. It is found by the present inventors unexpected that the cleavage sites of the proteases QA and QC of the present disclosure differ from that of the calf rennet, because the cleavage site of the calf rennet on κ-casein is Phe105-Met106 peptide linkage.

Method for Obtaining Asclepain of Asclepias Linn. and Cysteine Protease B of Calotropis R. Br.

In another aspect, the present disclosure in embodiments provides a method for obtaining asclepain of Asclepias Linn. and cysteine protease B of Calotropis R. Br., respectively. With the method in embodiments of the present disclosure, it is possible to extract the asclepain of Asclepias Linn. and the cysteine protease B of Calotropis R. Br. with high purity by effective and simple operations.

Referring to FIG. 1, the method in embodiments of the present disclosure includes steps of S100 and S200 as follows.

S100 Soaking

In the step S100, leaves of Cynanchum otophyllum Schneid. are soaked in a buffer, followed by collecting an extracted solution.

In embodiments of the present disclosure, the buffer is a citric acid-phosphate buffer. It is discovered by the present inventors that the proteases QA and QC exhibit high solubility in the citric acid-phosphate buffer among various buffers. Further, the C. otophyllum leaves are soaked in the citric acid-phosphate buffer by the present inventors, so as to extract the proteases QA and QC sufficiently. According to a preferable embodiment of the present disclosure, the citric acid-phosphate buffer is of a concentration of 10 mmol/L, thereby facilitating to extract the proteases QA and QC.

In embodiments of the present disclosure, the leaves of Cynanchum otophyllum Schneid. and the buffer are at a ratio of mass to volume from 1:10 to 1:30, for example 1:20. It is found by the present inventors that it is possible to extract proteases QA and QC from the C. otophyllum leaves sufficiently at such a suitable ratio of mass to volume, as well the proteases QA and QC are of a high concentration in an extracted solution.

It should be noted that term “a ratio of mass to volume” in this context refers to the ratio of the mass of C. otophyllum to the volume of the buffer.

In embodiments of the present disclosure, the leaves of Cynanchum otophyllum Schneid. are soaked under 4° C. to 25° C. for 30 minutes to 50 minutes, thereby facilitating to extract the proteases QA and QC from the C. otophyllum leaves sufficiently. In a preferable embodiment of the present disclosure, the C. otophyllum leaves are soaked under 4° C. for 40 minutes.

S200 purification

In the step S200, the extracted solution is purified, so as to obtain the asclepain of Asclepias Linn. and the cysteine protease B of Calotropis R. Br.

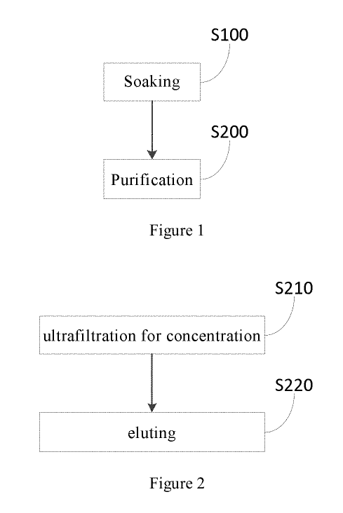

Referring to FIG. 2, purification in embodiments of the present disclosure includes steps of S210 and S220 as follows.

S210 Ultrafiltration for concentration

In the step S210, the extracted solution is subjected to ultrafiltration for concentration, thereby obtaining a concentrated solution.

In embodiments of the present disclosure, the ultrafiltration for concentration is performed using an ultrafiltration tube in 10.0 kD. It is found by the present inventors that a concentrated solution obtained by concentrating the extracted solution with the ultrafiltration tube in 10.0 kD contains proteins in a significant increased concentration, as well exhibits as three main bands in distinct molecule weights between 6.5 kD and 27.0 kD evidenced by the SDS-PAGE electrophoresis; while the filtered solution contains barely proteins. It is demonstrated that the proteases included in the extracted solution of C. otophyllum substantively contains proteases in a molecule weight more than 10.0 kD, thus it is possible to separate the proteases in the extracted solution of C. otophyllum from an impurity such as a small molecule effectively by using the ultrafiltration tube in 10.0 kD as an initial purification means.

S220 Elution

In the step S220, the concentrated solution is eluted on a chromatographic column, so as to obtain proteases of Cynanchum otophyllum Schneid.

In embodiments of the present disclosure, eluting the concentrated solution further includes steps of:

1) loading the concentrated solution onto the chromatographic column and collecting a first outflow, so as to obtain the asclepain of Asclepias Linn.;

2) loading a citric acid-phosphate buffer onto the chromatographic column obtained in step 1), with a second outflow obtained; and

3) loading a citric acid-phosphate buffer containing 0.6 mmol/L NaCl onto the chromatographic column obtained in step 2), and collecting a third outflow, so as to obtain the cysteine protease B of Calotropis R. Br.

It is found by the present inventors that the protease QA directly flows out rather than being absorbed onto the chromatographic column after the concentrated solution is loaded onto the chromatographic column, thereby obtaining the protease QA by collection of the first outflow; subsequently, the column is eluted with a citric acid-phosphate buffer without NaCl, thereby obtaining another C. otophyllum protease by collection of the second outflow; afterwards, the column is eluted with a citric acid-phosphate buffer containing 0.6 mmol/L NaCl again, during which the protease QC is desorbed from the column and flows out along with the buffer, thereby obtaining the protease QC by collection of the third outflow.

In embodiments of the present disclosure, the asclepain of Asclepias Linn. and the cysteine protease B of Calotropis R. Br. are those in the milk coagulant as defined in the above aspects. It should be understood by those skilled in the art that the features and advantages of the milk coagulant described above are also suitable for this method in embodiments, which will not be described again in detail.

Method of producing cheese

In still another aspect, the present disclosure in embodiments provides a method of producing cheese. According to embodiments of the present disclosure, the method includes: mixing cheese milk with the milk coagulant as defined in above aspects, thereby obtaining a mixture; and keeping the mixture standing for a time period, so as to obtain the cheese. As described above, the milk coagulant in embodiments of the present disclosure functions on milk clotting under a broad range of temperatures and at a wide range of pH values, as well exhibits great milk-clotting effect, with the cheese obtained in excellent texture and sensory quality.

It should be understood by the present inventors that term “cheese milk” in this context mainly refers to a raw material to be clotted.

In embodiments of the present disclosure, the method further includes: adjusting the mixture obtained to a pH value of 5.5 to 8.0. With adjustment of such the mixture obtained by mixing the cheese milk with the milk coagulant to be at an optimum pH value for milk-clotting (i.e. 5.5 to 8.0), it is beneficial for the milk coagulant to exhibit milk-clotting effect, thus obtaining cheese in excellent texture and sensory quality.

In embodiments of the present disclosure, the mixture is kept standing under 40° C. to 70° C. for 40 minutes to 90 minutes. With mixture standing under such the condition, it is beneficial for the milk coagulant to exhibit milk-clotting effect, thus obtaining cheese in excellent texture and sensory quality.

It should be understood by those skilled in the art that the features and advantages of the milk coagulant described above are also suitable for this method of producing cheese in embodiments, which will not be described again in detail.

Reference will be made in detail to examples of the present disclosure. It would be appreciated by those skilled in the art that the following examples are explanatory, and cannot be construed to limit the scope of the present disclosure. If the specific technology or conditions are not specified in the examples, a step will be performed in accordance with the techniques or conditions described in the literature in the art or in accordance with the product instructions. If the manufacturers of reagents or instruments are not specified, the reagents or instruments may be commercially available.

General procedure

1. Samples and Reagents

Samples of C. otophyllum Schneid. were collected from Jianchuan county (2,000 m above sea level) in Dali City, Yunnan province. After natural drying, the C. otophyllum samples were stored in a freezer at −20° C. until processing. Skim milk powder was obtained from Nouriz Dairy Co. (Shanghai, China); Q Sepharose Fast Flow from GE Healthcare (Uppsala, Sweden); calf rennet, Naturen Stamix 1150 NB, from Chr. Hansen (Hoersholm, Denmark); bromelain, papain, whole casein, α-CN, β-CN, κ-CN, and BSA from Sigma-Aldrich (St. Louis, Mo.); Coomassie Brilliant Blue G-250 and R-250 from Bio-Rad Laboratories (Hercules, Calif.); and 10-kDa ultrafiltration tubes from Millipore (Billerica, Mass.). All other chemicals were of analytical grade.

2. The protein concentrations of 4 partially purified protease extracted solutions were measured following the method of Bradford (1976) with Coomassie Brilliant Blue, where bovine serum albumin (BSA) was used as a protein standard.

3. Milk Clotting Activity Assay

The MCA of the C. otophyllum proteases was determined using a modified method of He et al. (2011). One milliliter of substrate (12% skim milk in 10 mM CaCl₂, pH 6.5) was incubated under 37° C. for 5 min, then 0.1 mL of the protease extracted solution was added. The time needed for curd formation was recorded and the MCA was expressed in Soxhlet units (SU). One SU of MCA was defined as the amount of the protease extracted solution required to clot 1 mL of substrate within 40 min at 37° C.

MCA =2400xV/ (vxt)

where V stands for the volume of the substrate (mL); v stands for the volume of the protease extract; and t stands for the milk-clotting time (s).

4. Caseinolytic Activity Assay

The caseinolytic activities of the C. otophyllum proteases were determined using a method modified from Mohanty et al. (2003). The substrate was prepared by dissolving 1% (wt/vol) of whole casein in 10 mmol/L of citric acidphosphate buffer (pH 6.5). The assay was performed by incubating 1.1 mL of substrate with 0.1 mL of partially purified protease extracted solution under 37° C. for 30 min and terminated using 1.8 mL of 5% (wt/vol) trichloroacetic acid (TCA). The blank control was prepared by adding the same amount of TCA to the protease extracted solution, then adding the substrate. After 30 min standing at room temperature, the completely precipitated proteins were removed by centrifuging at 5,000 × g for 20 min at room temperature. The protein content in the supernatant was then measured at 280 nm (UV-2102 PC, Unico Instrument Co. Ltd.). One unit of PA was defined as the amount of the protease extracted solution required for an increase of 0.01 in optical density in 1 min at 280 nm.

5. Kinetics Analysis of the κ-Casein by the Proteases

The hydrolysis kinetics of κ-casein by the proteases was evaluated as described by Nafi' et al. (2014) with some modifications. Five mg/mL of κ-casein stock solutions was dissolved in 10 mM citric acid-phosphate buffer (pH 6.5), then diluted with the buffer to concentrations of 0.1, 0.5, 1.0, 1.5, and 2.0 mg/mL. The diluted solutions were first incubated under 37° C. for 5 min, then the proteases were added at a ratio of 1 to 10 (vol/vol). After reacting for 10 min, the same volume of 5% (wt/vol) TCA was immediately added to terminate the reaction. The kinetic parameters, Michaelis constant (Km), catalytic turnover number (kcat), and proteolytic coefficient (kcat/Km), were calculated using the Lineweaver-Burk plot (Lineweaver and Burk, 1934).

6. Hydrolysis of Casein

The level of casein hydrolysis was determined as described by Huang et al. (2011). Bovine α-casein, β-casein, and κ-casein (50 mg each) were prepared separately by dissolving them in 10 mL of 10 mmol/L of citric acidphosphate buffer (pH 6.5). The proteases were added to each substrate at a ratio of 1:10 then hydrolyzed at 65° C. for 5, 15, and 30 min and 1, 2, and 4 h. The degree of degradation was analyzed using SDS-PAGE.

7. Statistical Analyses

All measurements were performed in triplicate. The data were analyzed by 1-way ANOVA (with Duncan's multiple range method) or the t-test using SPSS software (version 22.0, IBM, Armonk, N.Y.). The level for statistical significance was set at P<0.05.

Example 1

Basic Extraction

The C. otophyllum proteases were extracted as described by Huang et al. (2011) with some modifications. The C. otophyllum leaves were cut into pieces, mixed with a buffer at different solid-liquid ratios (i.e. ratio of mass to volume) for extraction, and then left for 2 h under 4° C., thus obtaining an extracted solution. After successive filtration through 4 layers of cheesecloth and a 0.22-μm membrane, the filtrate was concentrated by ultrafiltration with a 10-kDa molecular weight cut-off membrane at 8000 × g for 40 minutes at 4° C., with the retentate obtained as the concentrated solution of C. otophyllum proteases, which was assayed for protein concentration following the method of Bradford (1976) and curd formation time for 12% (w/v) skim milk.

1. Extraction Condition

Three aliquots of the C. otophyllum leaves and the buffer were individually incubated at 55° C., 25° C. or 4° C. for 40 minutes, and individual milk-clotting time was recorded, with results listed in Table 1 which demonstrate effect of different extraction conditions on milk-clotting activity of proteases of C. otophyllum leaves. It can be seen that the time required for curd formation upon treatment at 55° C. for 40 minutes is 7 times more than that upon treatment at 4° C. for 40 minutes, indicating the treatment at 55° C. for 40 minutes reduces the milk-clotting activity of proteases in C. otophyllum leaves. Thus, the C. otophyllum leaves are preferably incubated with the buffer at 4° C. for 40 minutes.

TABLE 1
Effect of different extraction conditions on extraction
of proteases of C. otophyllum leaves

| Different treatment temperatures | Milk-clotting time (min) |
|---|---|
| treatment at 55° C. for 40 minutes | 110 minutes |
| treatment at 25° C. for 40 minutes | 40 minutes |
| treatment at 4° C. for 40 minutes | 15 minutes |

2. Solid-Liquid Ratios

The C. otophyllum leaves and the buffer were individually mixed at different solid-liquid ratios of 1:10, 1:20 and 1:30. The increased solid-liquid ratio can improve the efficiency of protease extraction, but also decrease the concentrations of proteases extracted, thus affecting the stability and activity of the proteases. Summarizing the prior extraction processes, the solid-liquid ratios of 1:10, 1:20 and 1:30 were selected to investigate the effect on extraction of proteases of C. otophyllum leaves, with results listed in Table 2 which demonstrate effect of different solid-liquid ratios on milk-clotting activity of proteases of C. otophyllum leaves. It can be seen from the results that the concentrated solution of proteases of C. otophyllum leaves has a highest milk-clotting activity at the solid-liquid ratio of 1:20. Thus, the C. otophyllum leaves are preferably extracted at the solid-liquid ratio of 1:20.

TABLE 2
Effect of different solid-liquid ratios on extraction
of proteases of C. otophyllum leaves

| Solid-liquid ratio | Milk-clottting activity (U/mg) |
|---|---|
| 1:10 | 147.7 |
| 1:20 | 213.6 |
| 1:30 | 204.2 |

3. Buffers

The C. otophyllum leaves were mixed with ultrapure water and a citric acid-phosphate buffer containing 150 mmol/L NaCl, 1 mmol/L Cys and EDTA (pH 6.5, 10 mmol/L), respectively. It is found by the present inventors that the solubility of the C. otophyllum proteases increases in the presence of the salt at a low concentration, thus the saline-containing solution is utilized for extraction of the proteases of C. otophyllum leaves. Table 3 shows effect of different buffers on extraction of the proteases of C. otophyllum leaves. It can be seen from the results that the concentrated solution obtained with the citric acid-phosphate buffer (pH 6.5, 10 mmol/L) has a milk-clotting activity comparable to that obtained with the ultrapure water, whereas contains proteins more than twice of the proteins in the concentrated solution obtained with the ultrapure water, this is because the solubility of the C. otophyllum proteases is higher in the low-saline solution than the ultrapure water. Thus, the proteases of C. otophyllum leaves are preferably extracted with the citric acid-phosphate buffer.

TABLE 3
Effect of different buffers on extraction
of proteases of C. otophyllum leaves

| | Protein | Milk-clotting |
|---|---|---|
| Different buffers | content (mg/mL) | activity (U/mg) |
| ultrapure water | 0.136 | 198.1 |
| citric acid-phosphate buffer | 0.305 | 201.5 |
| (pH 6.5, 10 mmol/L) | | |

4. Separation and purification

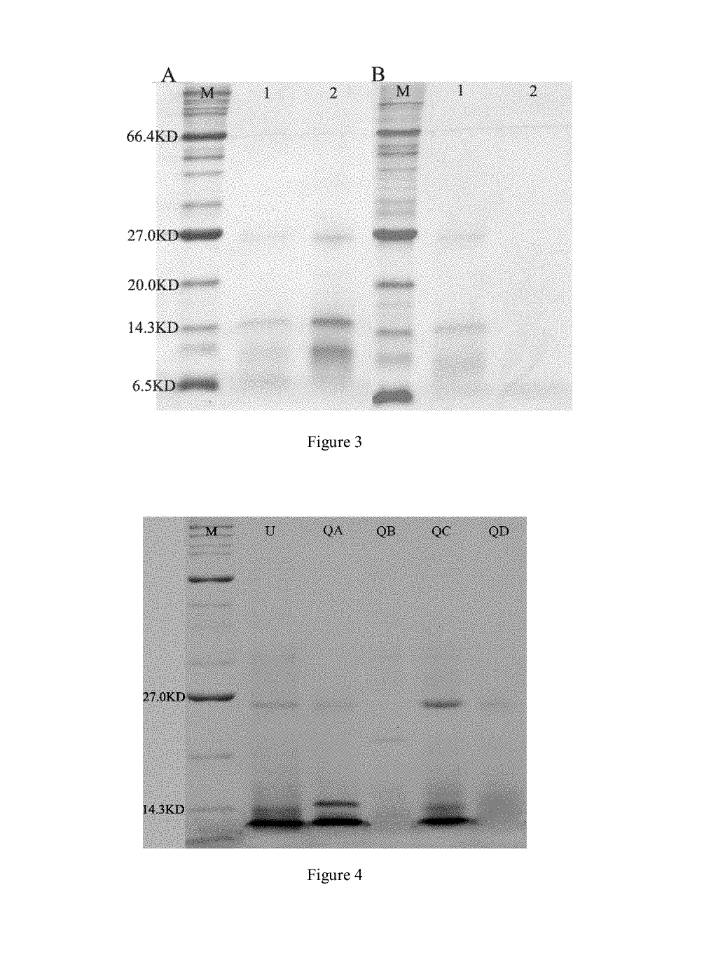

Ultrafiltration is a technology for separation of materials in different sizes by means of an ultrafiltration membrane. Generally, the material in a size less than the membrane pore size will pass through the membrane, while in contrast the material in a size above the membrane pore size will be cut off. Therefore, the ultrafiltration process enables to improve concentration of proteases in the extracted solution, as well to remove impurities (for example, some small molecules, such as phenols and pigments) in the extracted solution. FIG. 3 shows SDS-PAGE electrophoresis of a concentrated solution (A) and a filtered solution (B) of the C. otophyllum proteases. It can be seen that the concentrated solution obtained by concentrating the extracted solution with the ultrafiltration tube in 10.0 kD contains proteins in a significant increased concentration, as well exhibits three main bands in distinct molecule weights between 6.5 kD and 27.0 kD; while the filtered solution contains barely proteins. It is demonstrated that the proteases included in the extracted solution of C. otophyllum substantively contains proteases in a molecule weight more than 10.0 kD, thus it is possible to separate the proteases in the extracted solution of C. otophyllum from the impurity such as a small molecule effectively by using the ultrafiltration tube in 10.0 kD as an initial purification means.

The concentrated solution of C. otophyllum proteases obtained after ultrafiltration still contains various impurities (such as a protein), required to be further purified for separation of target proteins effectively. Further, the C. otophyllum proteases can be purified based on their different bonding abilities to weak anion exchange resins, so as to separate the target proteins from the impurities effectively. In specific, the retentate after the ultrafiltration was then applied to a E-C Polypropylene column (1.5×12 cm) packed with Q Sepharose Fast Flow, which had been equilibrated with 10 mmol/L of citric acid-phosphate buffer (pH 6.5). Elution for the proteases absorbed on the weak anion exchange resins was performed with gradient elute of 0, 0.6, and 1.0 mmol/L of NaCl (in binding buffer) to obtain 4 fractions of partially purified protease extracted solutions, i.e., QA (obtained by collection of a first outflow), QB (0 mmol/L), QC (0.6 mmol/L) and QD (1 mmol/L). All fractions were monitored at 280 nm using a UV detector (UV-2102 PC, Unico Instrument Co. Ltd., Shanghai, China) to detect the proteins. The protein concentration of each fraction was measured following the method of Bradford (1976), and the curd formation time for 12% (w/v) skim milk was determined; whereas the molecular weights of the fractions were determined using SDS-PAGE as described by Laemmli (1970). All the extraction and purification processes were carried out below 10° C. to protect the enzyme activity.

Table 4 shows the optical density at 280 nm, protein content and milk-clotting activity of the individual fractions. The results of the optical density at 280 nm and protein content show that the proteases are mainly concentrated in QA and QC fractions after purification of the concentrated solution derived by ultrafiltration with the weak anion exchange resins. From the results of curd formation time, only the QA and QC fractions enable the milk to clot.

TABLE 4
Identification of purified fractions of C. otophyllum proteases

| | | Protein | |
|---|---|---|---|
| Fraction | A280 | content (mg/ml) | curd formation time |
| QA | 0.382 | 0.189 | milk-clotting within 35 minutes |
| QB | 0.106 | 0.031 | no milk-clotting within 4 hours |
| QC | 0.425 | 0.155 | milk-clotting within 50 minutes |
| QD | 0.129 | 0.072 | no milk-clotting within 4 hours |

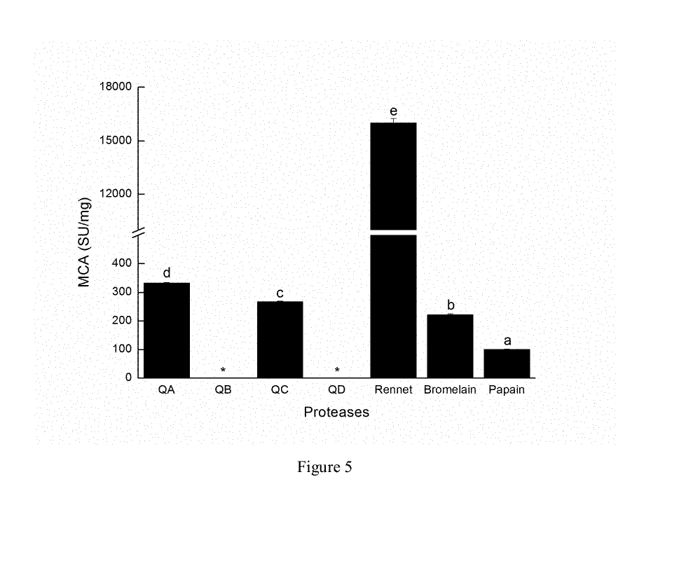

FIG. 4 shows SDS-PAGE electrophoresis of individual fractions (i.e. QA, QB, QC and QD) collected after elution with the weak anion exchange resins. It can be seen from the SDS-PAGE electrophoresis that the proteases are mainly concentrated in QA and QC, with molecular weights of about 14 and 27 kDa respectively, which are in consistent with the protein contents of the individual fractions measured by the method of Bradford (1976); while the QA and QC are estimated to have molecular weights of approximately 14.3 kD (analyzed to be 14.1 kD) and approximately 27.0 kD (analyzed to be 26.9 kD) respectively, therefore demonstrating that the weak anion exchange column is useful in effective purification of C. otophyllum proteases, with two proteases having milk-clotting activity but in different molecular weights.

Example 2 Basic characteristics of proteases QA and QC

Milk-Clotting Activity and Proteolytic Activity

Milk-clotting activity and proteolytic activity are very important properties for application of a milk coagulant. FIG. 5 shows that, only the QA and QC fractions enabled the milk to clot, which agreed with the electrophoresis results. The MCA values of QA and QC were 332.6 and 267.4 SU/mg, respectively. Because of the great variety of methods and different units used under different conditions found in the literature, it is difficult to compare the MCA of different proteases. Therefore, we also determined the MCA of calf rennet, bromelain, and papain as a comparison. FIG. 5 shows that although the MCA values of QA and QC were lower than that of calf rennet (16,000 SU/mg), they were significantly higher than those of bromelain (222.2 SU/mg) and papain (100.2 SU/mg). These results have shown that the QA and QC proteases extracted from C. otophyllum have the potential to be milk-clotting enzymes.

In addition to MCA, PA plays a critical role in evaluating the suitability of a milk-clotting enzyme. Proteolysis strongly affects the degradation patterns of caseins, which could further affect the yield and sensory properties of the cheese. Therefore an enzyme with a high MCA and low PA is preferred (Shah et al., 2014). Table 5 shows that the ratios of MCA/PA for QA and QC were 37.33 and 36.14, respectively, significantly higher values than those of purified enzymes from Onopordum acanthium (9.58), Bromelia hieronymi (4.18), and Philibertia gilliesii (4.82; Brutti et al., 2012).

TABLE 5
Milk-clotting activity and proteolytic
activity of C. otophyllum proteases

| | Milk-clotting | Proteolytic | |
|---|---|---|---|
| Proteases | activity (U/mg) | activity (SU/mg) | MCA/PA |
| QA | 332.6 ± 1.9^{b} | 8.91 ± 0.14^{b} | 37.33 |
| QC | 267.4 ± 2.7^{a} | 7.40 ± 0.21^{a} | 36.14 |

^{a,b}Means in a column with different superscripts are significantly different (P < 0.05).
Results are mean ± SD (n = 3). SU = Soxhlet unit.

Usually, the ratio of milk-clotting activity to proteolytic activity is a measurement for estimating whether the milk coagulant is suitable for cheese production. Higher the ratio often indicates more suitable the milk coagulant for the cheese production. As seen in Table 6, the ratios of milk-clotting activity to proteolytic activity of rennet derived from plants (such as Onopordum acanthium, Cynara cardunculus, Asclepias fruticosa, Bromeliaceae and the like) are all below 10.0, specifically the ratio of milk-clotting activity to proteolytic activity of Asclepias fruticosa is just 0.68, showing that all of these ratios are far lower than those of QA and QC (respectively, 37.37 and 36.14). Thus, the C. otophyllum proteases QA and QC are more suitable for cheese production.

TABLE 6
Milk-clotting activity and proteolytic
activity of rennet from other plants

| | | | ratio of Milk- |
|---|---|---|---|
| | Milk-clotting | Proteolytic | clotting activity |
| | activity | activity | to proteolytic |
| Type of plant rennet | (U/mL) | (SU/mL) | activity |
| Onopordum acanthium | 0.546 ± 0.004 | 0.019 ± 0.002 | 9.58 |
| Cynara cardunculus | 103.6 ± 4.1 | 16.4 ± 1.3 | 5.34 |
| Asclepias fruticosa | 0.7 ± 0.02 | 1.03 ± 0.06 | 0.68 |
| Bromelia balansae | 6.85 ± 0.3 | 1.32 ± 0.03 | 5.19 |
| Bromelia hieronymi | 10.0 ± 0.005 | 2.39 ± 0.04 | 4.18 |
| Philibertia gilliesii | 16.0 ± 0.003 | 3.32 ± 0.03 | 4.82 |

Kinetic Parameters of κ-Casein by the Proteases QA and QC

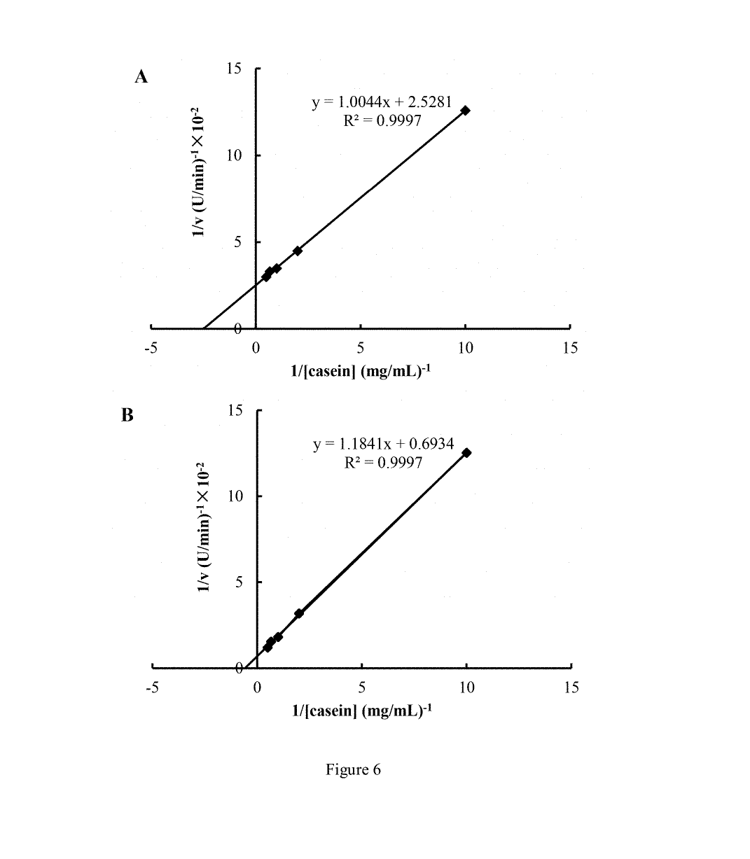

The higher proteolytic efficiency of chymosin on κ-casein, the faster generation of hydrophobic N-terminal moiety of κ-casein, the faster the aggregation rate of casein micelles (Shammet et al., 1992). Therefore, evaluation of kinetic parameters of the C. otophyllum proteases on κ-casein could provide better understanding of their milk clotting behaviors. The plot of 1/v versus 14S1 for the hydrolysis of κ-casein by the C. otophyllum proteases is shown in FIG. 6, with the kinetic parameters calculated from the plots shown in Table 7. Km is the concentration of substrate required to produce 50% of the maximum velocity value. A lower Km indicates a high enzyme affinity to the substrate when the substrate concentration is low, whereas a higher Km indicates a high enzyme affinity to the substrate only when the substrate concentration is higher (He et al., 2011). The Kcat is the catalytic center activity, and the ratio kcat/Km is the proteolytic coefficient, with a high kcat/Km indicating a high enzyme proteolytic efficiency on κ-casein (Vreeman et al., 1986). The Km of QC (1.708 mg/mL) was more than 4 times that of QA (0.397 mg/mL), whereas the kcat/Km of QC was one-fifth that of QA (251.01 vs. 48.95 mL/mg.min), indicating a significantly higher enzyme affinity and proteolytic efficiency for QA than QC. The Km values of the C. otophyllum proteases QA at 37° C. were consistent with those of ginger proteases at 40° C. (0.237-0.359 mg/mL; Huang et al., 2011).

TABLE 7
Kinetic parameters of Cynanchum otophyllum
Schneid. proteases QA and QC¹.

| Proteases | QA | QC |
|---|---|---|
| Kₘ (mg/mL) | 0.397 ± 0.002^{a} | 1.708 ± 0.014^{b} |
| k_{cat} (min) | 99.65 ± 2.32^{b} | 83.61 ± 1.46^{a} |
| k_{cat}./Kₘ [mL/(mg · min)] | 251.01 ± 6.11^{b} | 48.95 ± 1.93^{a} |

^{a-b}Means in a column with different superscripts are significantly different (P < 0.05).
¹Results are mean ± SD (n = 3). Michaelis constant (Km), catalytic turnover number (Kcat), and proteolytic coefficient (kcat/Km).

Identification of the Proteases QA and QC

The purified C. otophyllum proteases were subjected to strip identification, with results shown in Table 8 which are consistent between those from NCBI and Uniprot data banks. It has not yet reported study on C. otophyllum proteases, thus protein identification of such the proteases contributes to general knowledge of their enzymatic properties. It can be seen from Table 8 that the protein scores of the QA and QC are significantly higher than an acceptable score threshold of 65, therefore the QA is identified to be asclepain of Asclepias Linn. and the QC is identified to be cysteine protease B of Calotropis R. Br.

TABLE 8
Identification results of C. otophyllum QA and QC by mass spectrometry

| C. otophyllum | Accession | | | Protein | Sequence | Molecular |
|---|---|---|---|---|---|---|
| proteases | number ^{a} | Protein name ^{b} | Plant source ^{c} | score^{d} | coverage ^{e} | weight ^{f} |
| QA | gi\|215414308 | asclepain cI | Asclepias curassavica | 150.73 | 11.3% | 21.3 kD |
| | gi\|215414310 | asclepain cII | Asclepias curassavica | 93.46 | 7.3% | 20.9 kD |
| QC | gi\|615503249 | procerain B | Calotropis procera | 267.53 | 53.4% | 36.4 kD |
| | gi\|475638275 | procerain B, | Calotropis procera | 286.56 | 53.3% | 23.8 kD |
| | | partial | | | | |

Noted:
^{a} Accession number in NCBI data bank;
^{b} Protein name obtained from NCBI data bank;
^{c} Source of the protein from NCBI data bank;
^{d}molecular weight searching score of protein;
^{e} Sequence coverage of identified protein;
^{f} theoretical molecular weight from Uniprot.

Specificity of the Proteases QA and QC on the Hydrolysis of Types of Casein

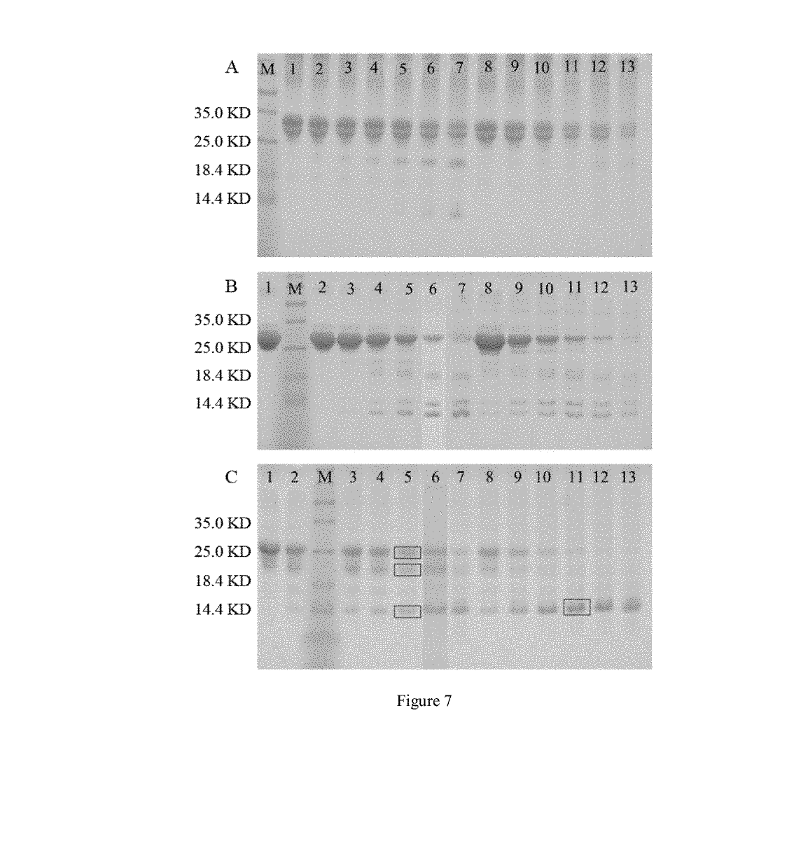

FIG. 7 compares the specificity of the C. otophyllum proteases on the hydrolysis of isolated α-casein, β-casein, and κ-casein as a function of time at 60° C. The results showed that C. otophyllum proteases could degrade all 3 caseins, with obvious degradation observed after 30 min β-Casein and κ-casein were completely hydrolyzed after 4 h of incubation, whereas α-casein was only partially hydrolyzed. In addition, the peptides in all caseins degraded and generated diffuse bands showing those of low molecular weight at the bottom of the gel as the reaction time increased. The number of breakdown products for α-casein, β-casein, and κ-casein, was 2, 3, and 1, respectively (See, lane 7 in A, B and C). The QC also exhibited a higher rate of hydrolysis than QA for β-casein and κ-casein but was less for α-casein. Most of the κ-casein was hydrolyzed by QC after 30 min of incubation, but hydrolysis by QA required almost 4 h. Previous studies have shown that the cysteine protease actinidin, and the protease from Dregea sinensis Hemsl. could also completely degrade β-casein and κ-casein and partially degrade α-casein (Lo Piero et al., 2011; Zhang et al., 2015).

Example 3 Effect on Enzymatic Property—proteolytic activity

1. pH value

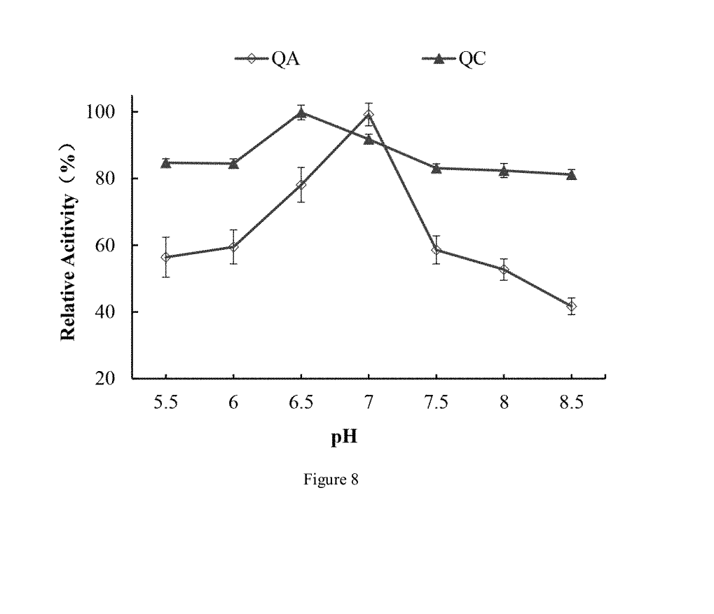

A variation of pH in the system can affect the dissociation of the dissociable groups in the active center and thus affect the proteolytic activity (Shah et al., 2014). The effect of pH on the PA of the C. otophyllum proteases was investigated to reveal their proteolytic properties. It is shown in FIG. 8 that the C. otophyllum proteases QA and QC both increased at first then significantly decreased as the pH increased. The PA of protease QA reached an optimum value at pH 7, with over 40% of residual activity retained over the whole pH range. The optimum pH value for QC was slightly lower (pH 6.5), and exhibited a significantly higher residual activity of over 80% over the whole pH range (FIG. 8).

As milk has a natural pH of 6.5 to 6.7 and the coagulation of milk cake is usually performed at a pH of 5.5 to 6.0, the fact that C. otophyllum proteases can maintain PA under neutral and alkaline conditions make them suitable for producing milk cake.

2. Temperature

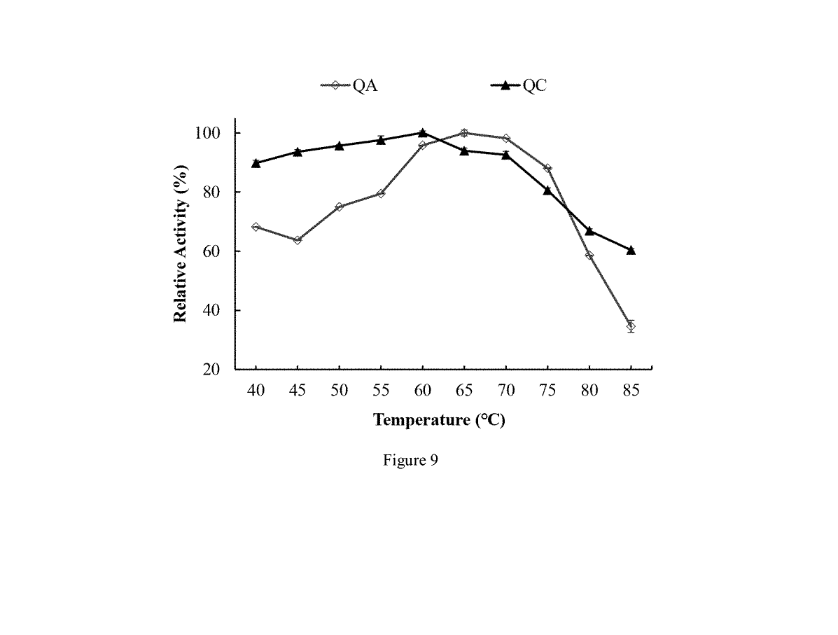

The effect of temperature on the PA of the C. otophyllum proteases was investigated to reveal their proteolytic properties (PA). FIGS. 9 shows the effect of temperature on caseinolytic activity of the C. otophyllum proteases QA and QC, which exhibit similar proteolytic patterns for the temperature dependence of PA as for the pH value. The proteases QA and QC respectively reach the optimum value at 65° C. and 60° C., and exhibit a broad optimum temperature range between 40 and 70° C. for the hydrolysis of whole casein. The QA and QC also still maintained about 60% residual activity when the temperature was increased to 80° C., indicating that C. otophyllum proteases were highly resistant to temperature. However, the PA of the proteases QA and QC drops rapidly at a high temperature such as above 70° C., probably because the high temperature leads to thermal denaturation of proteases and thus results in enzyme deactivation.

The pH and temperature profiles were similar to those of the milk clotting enzymes from C. trigonus Roxburghi and S. dubium (Asif-Ullah et al., 2006; Ahmed et al., 2009).

3. Protease Inhibitor

The type of protease was determined as described by Mazorra-Manzano et al. (2013) with modification.Different protease inhibitors (8 mmol/L, 0.1 mL), a serine protease (phenylmethylsulfonyl fluoride, PMSF), a cysteine protease [transepoxy-succinylleucyl-amido-(4-guanidino)-butane, E-64], a metallo protease (EDTA), and an aspartic protease (pepstatin A), were added to 1 mL of the proteases. The mixtures were incubated at 37° C. for 30 min, then PA was evaluated. The percentage inhibition was calculated as follows:

percentage inhibition=100−[100−(residual activity/activity without inhibitor)].

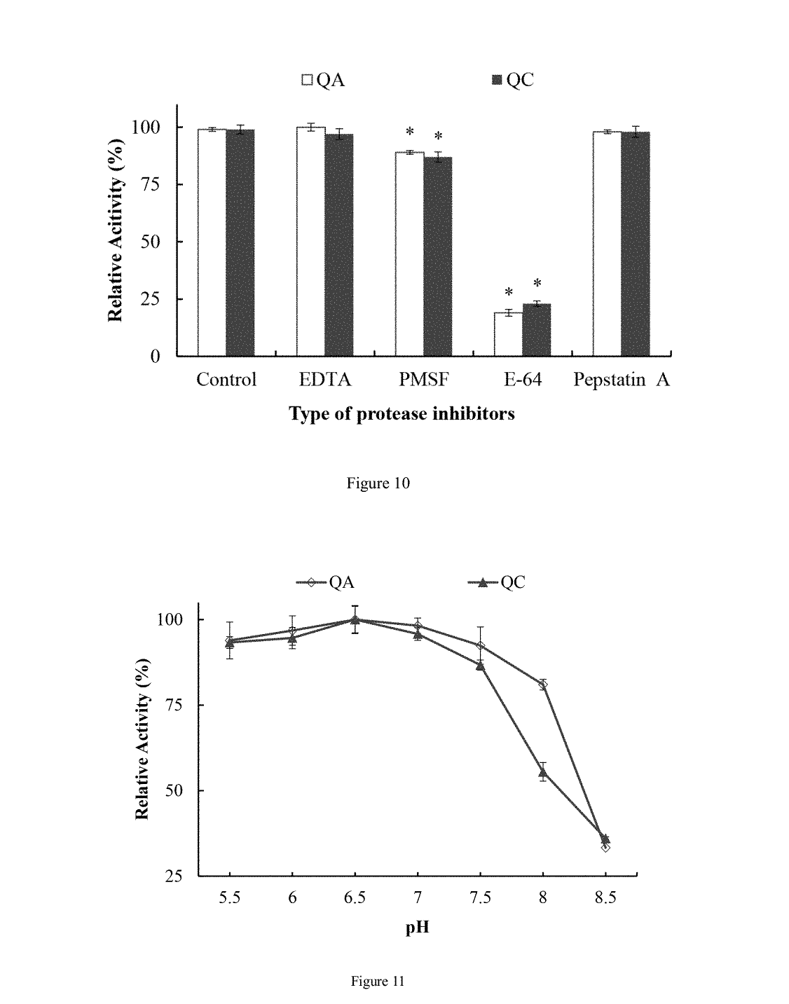

FIGS. 10 shows the effect of such four protease inhibitors on proteolytic activity of the C. otophyllum proteases QA and QC, with similar proteolytic patterns for the QA and QC. The effect of metallo protease EDTA and aspartic protease pepstatin A does not affect the PA of the proteases, whereas cysteine protease (transepoxy- succinylleucyl- amido-(4-guanidino)-butane, E-64) dramatically inhibited their activity compared to the control (where PA of the QA and QC drops to 20% of the control) (P<0.05), indicating that the C. otophyllum proteases were most likely to be a cysteine protease. Proteases purified from ginger, B. hieronymi fruits, and D. sinensis have also been shown to be a cysteine protease (Bruno et al., 2010; Nafi' et al., 2014; Zhang et al., 2015). Cysteine proteases, such as papain, bromelain, ficin, and calotropins, have been widely used in dairy processing (Sharma et al., 2012).

Compared to the control, the activity of QA and QC was also inhibited by serine protease Phenylmethylsulfonyl fluoride (PMSF), by showing about 15% reduction in the activity (FIG. 10). The activity of cysteine proteases extracted from ginger, garlic, and capsules of caper (Capparis spinosa) have all been found to be partially inhibited by PMSF (Parisi et al., 2002; Demir et al., 2008; Nafi'et al., 2013). The reduction of protease activity was most probably caused by PMSF bound to serine with alanine, phenylalanine, or tryptophan residues on the non-active side of the C. otophyllum proteases, thereby reducing the affinity of the substrate with the enzyme and therefore the enzyme activity (Nafi' et al., 2014).

In view of the above, it is indicated that the C. otophyllum proteases QA and QC contain active centers of serine protease and cysteine proteinase.

Example 4 Effect on Enzymatic Property—milk-clotting activity

1. pH

FIG. 11 shows that the MCA of the C. otophyllum proteases QA and QC exhibited a similar trend as the pH varied from 5.5 to 8.5, with the optimum pH between 5.5 and 7.5. It can be seen from FIG. 11 that the milk-clotting activity of the QA changes unobvious within a pH range between 5.5 and 7.5, but decreases rapidly at a pH value above 7.5, with a relative MCA of 30% recorded when the pH was raised to 8.5, indicating significant reduce of the milk-clotting activity of the QA under an alkaline condition (P<0.05); while the QC has a similar pH-activity profile as the QA, with a relative MCA of 30% recorded when the pH was raised to 8.5, indicating significant reduce of the milk-clotting activity of the QC under an alkaline condition. Thus, an increased pH value of the skim milk results in a decreased milk-clotting activity, with increased milk-clotting time observed under the alkaline condition. This loss of activity under alkaline conditions may be caused by interference from casein aggregation or irreversible changes in the conformation of the casein (Hashem, 2000; Home and Banks, 2004).

2. Temperature

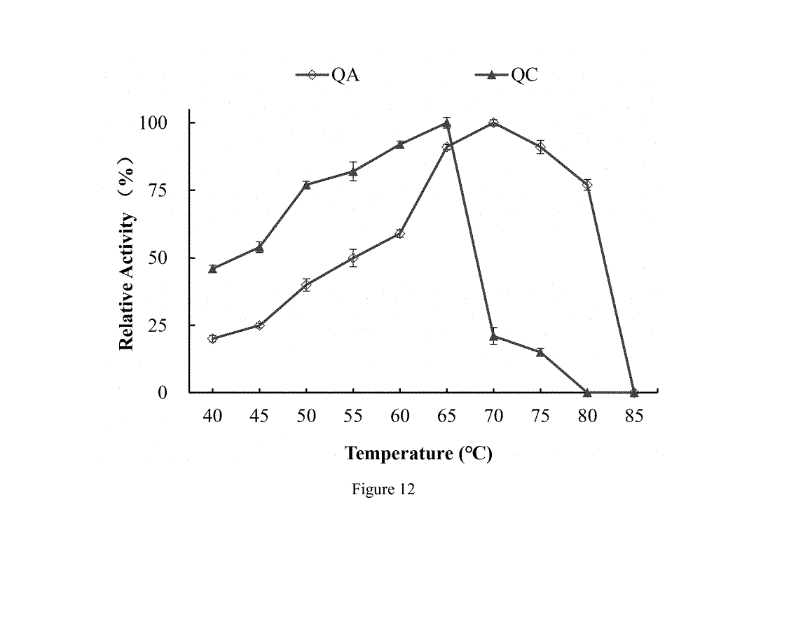

The temperature also has a significant influence on C. otophyllum proteases. As the temperature increased from 40 to 85° C., the MCA of the QA and QC proteases first increased significantly, then decreased sharply, and finally fell to zero (FIG. 12). However, the optimum temperatures for QA and QC were different (65-80° C. vs. 50-65° C., respectively). The MCA of QA peaked at 70° C. at a value of 100%, and remains a high milk-clotting activity (i.e. approximately 80% of the maximum) at a temperature between 75° C. and 80° C.; while in contrast no milk-clot formation of the skim milk is observed during milk-clotting at a temperature of 85° C., demonstrating the QA is completely inactivated at a temperature above 80° C. The MCA of QC peaked at 65° C. at a value of 100%, but decreased rapidly at a temperature above 65° C. (with 20% of the maximum), demonstrating the QA exhibits a better ability to resist heat than QC.

Heating may have denatured the whey proteins to form a complex of κ-casein with whey protein, which decreased the effective κ-casein concentration in the substrate and thus increased the curding time (Horne and Banks, 2004). The optimum temperature observed in the C. otophyllum proteases was similar to that of the milk-clotting enzyme from ginger (65° C.; Huang et al., 2011) and from melon (70° C.; Mazorra-Manzano et al., 2013), but was significantly higher than that for calf rennet (40-42° C.; Horne and Banks, 2004), probably due to differences in enzyme structure. As the milk clotting for traditional milk cake is usually performed at a temperature of about 65 to 80° C. with a pH of about 5.5 to 6.0, the C. otophyllum proteases are thus suitable for producing milk cake.

3. Metal Ion

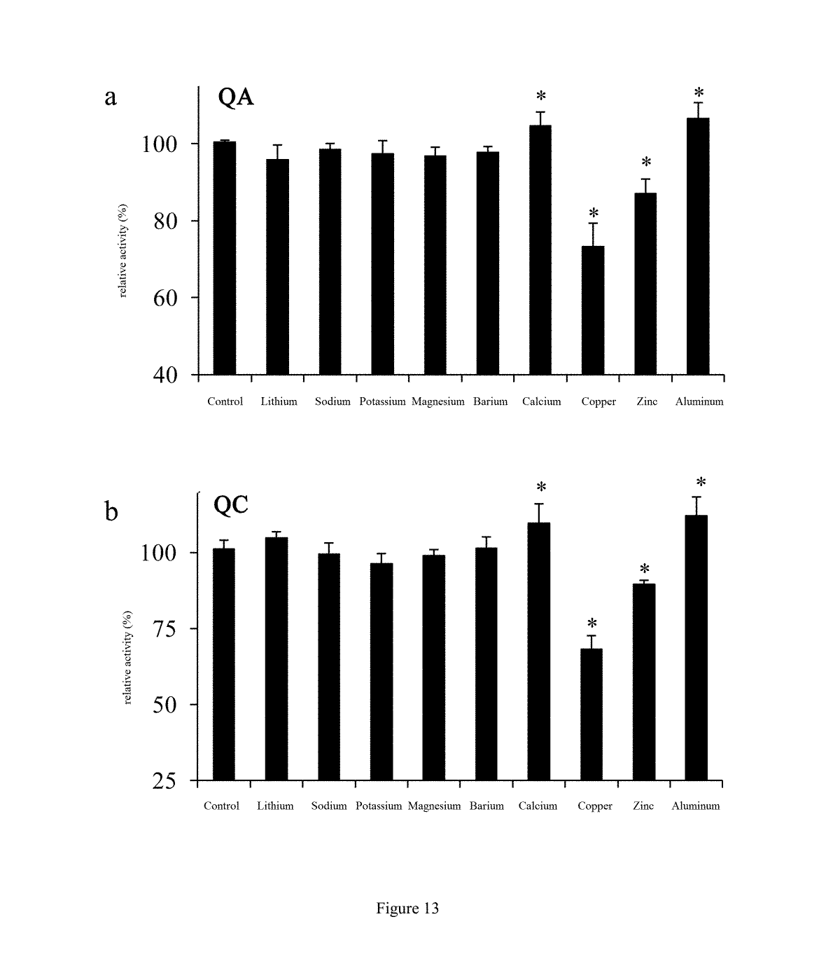

Some metal ions as a cofactor can participate in the milk-clotting reaction, thus affecting activity of enzyme. Further, the skim milk is of increased iron strength after addition of salts, thus interfering the stability of casein micelle and the formation of the milk clot. FIG. 13 shows milk-clotting activities of C. otophyllum proteases QA and QC determined after addition of different metal ions (10 mmol/L) to 12% (w/v) skim milk (containing 10 mmol/L CaCl₂, pH 6.5) respectively, with consistent results for the QA and QC which indicate effects of different metal ions on milk-clotting activity. It can be seen from FIG. 10 that addition of lithium, sodium, potassium, magnesium or barium ions to the skim milk exhibits no significant effect on milk-clotting activities of the QA and QC (P>0.05); while addition of copper or zinc ions exhibits significant inhibition on milk-clotting activities of the QA and QC (P<0.05), but addition of calcium or aluminium ions exhibits a significant increased effect on milk-clotting activities of the QA and QC (P<0.05), contrary to the copper or zinc ions. The reason of the promotion on milk coagulant activity and the decrease in milk-clotting time by the calcium ions is that they combines with exposed α-casein and β-casein during aggregation of the casein (i.e. the second step of the milk-clotting reaction), so as to promote formation of the milk clot. Further, it has been investigated that the calcium ions protect enzyme, especially those being poor resistant to temperature, and stabilizes the structure of enzyme, thus an amount of CaCl₂ is often added to protect the structure of enzyme during cheese production, for increasing activity and speeding milk-clotting.

Example 5 Analysis of the Cleavage Site on x-Casein by the Proteases

The cleavage site on κ-casein used by the proteases was determined as described by Zhang et al. (2015) with some modifications. The κ-casein solution was prepared in 10 mmol/L of citric acid-phosphate buffer (pH 6.5) at a concentration of 5 mg/mL. The C. otophyllum proteases QA and QC each were mixed with the κ-casein solution individually at a ratio of 1:10 and then incubated for 1 hour at 60° C. to completely ensure the milk was clotted. The mixture was mixed with the loading buffer in a 95° C. water bath for 5 minutes and then separated using urea SDS-PAGE. The target product bands were excised from the stained gel and then in-gel digested by trypsin overnight at 37° C. The digested solution obtained were lyophilized and then analyzed using an Orbitrap MS (LTQ Orbitrap XL, Thermo Fisher Scientific, San Jose, Calif.). Table 8 shows amino acid sequences of peptides primarily obtained (SEQ IN NOs: 1-7).

After 1 h of incubation, the κ-casein hydrolysate was excised from the gel to determine the cleavage site on κ-casein by the C. otophyllum proteases QA and QC, as indicated in the frames of FIG. 7C. The peptide sequences obtained of the in-gel digests of peptide segments from κ-casein are shown in Table 8. Four peptides from the hydrolysate of QA were matched to κ-casein with a molecular weight searching (MOWSE) score of 729.38 and three peptides from the hydrolysate of QC with a MOWSE score of 988.79. The high MOWSE scores for the C. otophyllum proteases QA and QC indicated an effective identification for the N-terminal moiety of κ-casein. The omission of some peptide sequences from the results may have been caused by the further hydrolysis of κ-casein into small peptides, which could not be detected by the Orbitrap analysis. Moreover, it is well known that trypsin is preferentially cleaved at Arg and Lys in position P1 (Keil, 2012). Therefore, by eliminating the peptides obtained from trypsin and the overlapped peptides, we can conclude that the primary cleavage site of C. otophyllum proteases QA and QC on κ-casein was Ser132-Thr133. κ-casein was hydrolyzed by QA into 2 peptides, κ-casein (f1-132) with a molecular weight of 15,139.72 Da and κ-casein (f133-169) with a molecular weight of 3,840.89 Da; whereas by QC into 3 peptides, κ-casein (f1-14) with a molecular weight of 1,743.78 Da, 78 -casein (f15-132) with a molecular weight of 13,413.94 Da and κ-casein (f133-169) with a molecular weight of 3,840.89 Da. This result agreed with the molecular weight observed from the electrophoresis results (˜15 kDa, FIG. 6C). The cleavage sites of the C. otophyllum proteases QA and QC in κ-casein differed from those of calf rennet, which cleaved at Phe105-Met106 and others reported for plant proteases: Ala90-Glu91, His102-Leu103, and Thr121-Ile122 for ginger protease (Huang et al., 2011); Phe105-Met106, Arg97-His98, Lys111-Lys112, or Lys112-Asn113 for lettuce protease (Lo Piero et al., 2002); and Phe105-Met106 and Lys116-Thr117 for proteases purified from sunflower and albizia seeds (Egito et al., 2007).

TABLE 8
Identification of peptide sequences of in-gel digested product of peptide segments from
κ-casein by C. otophyllum Schneid. proteases QA and QC using Orbitrap MS

| | | | Molecular weight of | | |
|---|---|---|---|---|---|
| | | | peptide (Da) | | |
| | | | Observed | | Peptide |
| Protease | Score | Peptide sequence | ([M + H]⁺)¹ | Calculated | origin |
| QA | 729.38 | SPAQILQWQVLSNTVPAK (SEQ ID NO: 1) | 1980.08 | 1979.08 | κ-casein |
| | | | | | (69-86) |
| | | SCQAQPTTMAR (SEQ ID NO: 2) | 1192.56 | 1192.53 | κ-casein |
| | | | | | (87-97) |
| | | HPHPHLSFMAIPPK (SEQ ID NO: 3) | 1608.85 | 1607.84 | κ-casein |
| | | | | | (98-111) |
| | | NQDKTEIPTINTIASGEPTS (SEQ ID NO: 4) | 2116.04 | 2115.03 | κ-casein |
| | | | | | (113-132) |
| QC | 988.79 | PAAVRSPAQILQWQVLSNTVPAKSCQAQP | 3648.94 | 3647.90 | κ-casein |
| | | TTMAR (SEQ ID NO:5) | | | (63-97) |
| | | HPHPHLSFMAIPPK (SEQ ID NO: 6) | 1608.84 | 1607.84 | κ-casein |
| | | | | | (98-111) |
| | | NQDKTEIPTINTIASGEPTS (SEQ ID NO: 7) | 2116.04 | 2115.03 | κ-casein |
| | | | | | (113-132) |

gi | 162811 κ-casein precursor (Bos Taurus); Mass: 21255.89 Da. Orbitrap, Thermo Fisher Scientific (San Jose, CA).
¹[M + H]⁺indicates the mass and the charge of the molecular ions.

In the specification of the present disclosure, the terms “an embodiment”, “some embodiments”, “an example”, “a specific example”, “some examples” or “a particular embodiment” and the like are intended to refer to particular features, structures, materials or characteristics described by way of example or embodiment are contained in at least one embodiment or example of the disclosure. In this specification, the schematic representation of the above terms does not necessarily refer to the same embodiment or example Moreover, the particular features, structures, materials or characteristics described may be combined in any suitable manner in one or more embodiments or examples. In addition, various embodiments or examples described in the specification, as well as features of such the embodiments or examples, may be combined by those skilled in the art without conflict.

Although embodiments of the present disclosure have been described, it will be understood by those skilled in the art that such the embodiments are explanatory and should not be construed to limiting the present disclosure. Further, various changes, modifications, substitutions and variations can be made in these embodiments by those skilled in the art without departing from the scope of the present disclosure.

### REFERENCES

1. Ahmed, I. A. M., I. Morishima, E. E. Babiker, and N. Mori. 2009. Dubiumin, a chymotrypsin-like serine protease from the seeds of Solanum dubium Fresen. Phytochemistry 70:483-491.

2. Asif-Ullah, M., K.-S. Kim, and Y. G. Yu. 2006. Purification and characterization of a serine protease from Cucumis trigonus Roxburghi. Phytochemistry 67:870-875.

3. Bruno, M. A., C. M. Lazza, M. E. Errasti, L. M. I. Lopez, N. 0. Caffini, and M. F. Pardo. 2010. Milk clotting and proteolytic activity of an enzyme preparation from Bromelia hieronymi fruits. Lebensm. Wiss. Technol. 43:695-701.

4. Brutti, C. B., M. F. Pardo, N. 0. Caffini, and C. L. Natalucci. 2012. Onopordum acanthium L. (Asteraceae) flowers as coagulating agent for cheesemaking. Lebensm. Wiss. Technol. 45:172-179.

5. Demir, Y., A. A. Gungor, E. D. Duran, and N. Demir. 2008. Cysteine protease (capparin) from capsules of caper (Capparis spinosa). Food Technol. Biotechnol. 46:286-291.

6. Egito, A. S., J. M. Girardet, L. E. Laguna, C. Poirson, D. Mollé, L. Miclo, G Humbert, and J. L. Gaillard. 2007. Milk-clotting activity of enzyme extracts from sunflower and albizia seeds and specific hydrolysis of bovine x-casein. Int. Dairy J. 17:816-825.

7. He, X., F. Ren, H. Guo, W. Zhang, X. Song, and B. Gan. 2011. Purification and properties of a milk-clotting enzyme produced by Bacillus amyloliquefaciens D4. Korean J. Chem. Eng. 28:203-208.

8. Huang, X. W., L. J. Chen, Y. B. Luo, H. Y. Guo, and F. Z. Ren. 2011. Purification, characterization, and milk coagulating properties of ginger proteases. J. Dairy Sci. 94:2259-2269.

9. Lineweaver, H., and D. Burk. 1934. The determination of enzyme dissociation constants. J. Am. Chem. Soc. 56:658-666.

10. Lo Piero, A. R., I. Puglisi, and G Petrone. 2002. Characterization of “lettucine,” a serine-like protease from Lactuca sativa leaves, as a novel enzyme for milk clotting. J. Agric. Food Chem. 50:2439-2443.

11. Lo Piero, A. R., I. Puglisi, and G Petrone. 2011. Characterization of the purified actinidin as a plant coagulant of bovine milk. Eur. Food Res. Technol. 233:517-524.

12. Mazorra-Manzano, M. A., T. C. Perea-Gutierrez, M. E. Lugo-Sanchez, J. C. Ramirez-Suarez, M. J. Torres-Llanez, A. F. Gonzalez-Cordova, and B. Vallejo-Cordoba. 2013. Comparison of the milk-clotting properties of three plant extracts. Food Chem. 141:1902-1907.

13. Mohanty, A. K., K. Mukhopadhyay, J. K. Kaushik, S. Grover, and V. K. Batish. 2003. Isolation, purification and characterization of chymosin from riverine buffalo (Bubalos bubalis). J. Dairy Res. 70:37-43.

14. Nafi', A., H. L. Foo, B. Jamilah, and H. M. Ghazali. 2013. Properties of proteolytic enzyme from ginger (Zingiber officinale Roscoe). Int. Food Res. J. 20:363-368.

15. Nafi', A., F. H. Ling, J. Bakar, and H. M. Ghazali. 2014. Partial characterization of an enzymatic extract from Bentong ginger (Zingiber officinale var. Bentong). Molecules 19:12336-12348.

16. Parisi, M., S. Moren, and G Fernandez. 2002. Characterization of a novel cysteine peptidase from tissue culture of garlic (Allium sativum L.). In Vitro Cell. Dev. Pl. 38:608-612.

17. Shah, M. A., S. A. Mir, and M. A. Paray. 2014. Plant proteases as milk-clotting enzymes in cheesemaking: A review. Dairy Sci. Technol. 94:5-16.

18. Shammet, K. M., R. J. Brown, and D. J. McMahon. 1992. Proteolytic activity of some milk-clotting enzymes on x-casein. J. Dairy Sci. 75:1373-1379.

19. Sharma, A., M. Kumari, and M. Jagannadham. 2012. Religiosin C, a cucumisin-like serine protease from Ficus religiosa. Process Biochem. 47:914-921.

20. Vreeman, H. J., S. Visser, C. J. Slangen, and J. Van Riel. 1986. Characterization of bovine lc-casein fractions and the kinetics of chymosin-induced macropeptide release from carbohydrate-free and carbohydrate-containing fractions determined by high-performance gel-permeation chromatography. Biochem. J. 240:87-97.

21. Zhang, Y., H. Wang, L. Tao, and A.-X. Huang. 2015. Milk-clotting mechanism of Dregea sinensis Hemsl. protease. J. Dairy Sci. 98:8445-8453.

What is claimed is:

1. A milk coagulant, comprising at least one of asclepain of Asclepias Linn. and cysteine protease B of Calotropis R. Br.

2. The milk coagulant according to claim 1, wherein the asclepain of Asclepias Linn. and the cysteine protease B of Calotropis R. Br. are from Cynanchum otophyllum Schneid.

3. The milk coagulant according to claim 1, wherein the asclepain of Asclepias Linn. and the cysteine protease B of Calotropis R. Br. are from leaves of the Cynanchum otophyllum Schneid.

4. The milk coagulant according to claim 1, further comprising at least one of a calcium-containing compound and an aluminum-containing compound.

5. The milk coagulant according to claim 1, wherein the milk coagulant functions under a temperature of 40° C. to 70° C. and at a pH value of 5.5 to 8.0.

6. The milk coagulant according to claim 1, wherein the asclepain of Asclepias Linn. is capable of hydrolyzing Ser132-Thr133 peptide linkage on κ-casein.

7. The milk coagulant according to claim 1, wherein the cysteine protease B of Calotropis R. Br. is capable of hydrolyzing Asp14-Glu15 peptide linkage and Ser132-Thr133 peptide linkage on κ-casein.

8. A method for obtaining asclepain of Asclepias Linn. and cysteine protease B of Calotropis R. Br., comprising:
soaking leaves of Cynanchum otophyllum Schneid. in a buffer, followed by collecting an extracted solution; and
purifying the extracted solution, so as to obtain the asclepain of Asclepias Linn. and the cysteine protease B of Calotropis R. Br., respectively,
wherein the asclepain of Asclepias Linn. and the cysteine protease B of Calotropis R. Br. are those in the milk coagulant as defined in claim 1.

9. The method according to claim 8, wherein the buffer is a citric acid-phosphate buffer.

10. The method according to claim 9, wherein the citric acid-phosphate buffer is of a concentration of 10 mmol/L.

11. The method according to claim 8, wherein the leaves of Cynanchum otophyllum Schneid. and the buffer are at a ratio of mass to volume from 1:10 to 1:30.

12. The method according to claim 8, wherein the leaves of Cynanchum otophyllum Schneid. and the buffer are at a ratio of mass to volume from 1:20.

13. The method according to claim 8, wherein the leaves of Cynanchum otophyllum Schneid. are soaked under 4° C. to 25° C. for 30 minutes to 50 minutes.

14. The method according to claim 8, wherein the leaves of Cynanchum otophyllum Schneid. are soaked at 4° C. for 40 minutes.

15. The method according to claim 8, wherein purifying the extracted solution further comprises:
subjecting the extracted solution to ultrafiltration for concentration, thereby obtaining a concentrated solution; and
eluting the concentrated solution on a chromatographic column, so as to obtain proteases of Cynanchum otophyllum Schneid., wherein
eluting the concentrated solution further comprises steps of:
1) loading the concentrated solution onto the chromatographic column and collecting a first outflow, so as to obtain the asclepain of Asclepias Linn.;
2) loading a citric acid-phosphate buffer onto the chromatographic column obtained in step 1), with a second outflow obtained; and
3) loading a citric acid-phosphate buffer containing 0.6 mmol/L NaCl onto the chromatographic column obtained in step 2), and collecting a third outflow, so as to obtain the cysteine protease B of Calotropis R. Br.

16. The method according to claim 15, wherein the ultrafiltration for concentration is performed using an ultrafiltration tube in 10.0 kD.

17. A method of producing cheese, comprising:
mixing cheese milk with the milk coagulant as defined in claim 1, thereby obtaining a mixture; and
keeping the mixture standing for a time period, so as to obtain the cheese.

18. The method according to claim 17, further comprising:
adjusting the mixture obtained to a pH value of 5.5 to 8.0; and
keeping the mixture standing at 40° C. to 70° C. for 40 minutes to 90 minutes.
